(19) United States
(12) Patent Application Publication
(10) Pub. No.: US 2023/0404506 A1
(43) Pub. Date: Dec. 21, 2023

(54) RADIATION IMAGING APPARATUS, INFORMATION PROCESSING APPARATUS, INFORMATION PROCESSING METHOD, AND NON-TRANSITORY COMPUTER-READABLE STORAGE MEDIUM

(71) Applicant: CANON KABUSHIKI KAISHA, Tokyo (JP)

(72) Inventors: Kosuke TERUI, Kanagawa (JP); Atsushi IWASHITA, Tokyo (JP); Ryuichi FUJIMOTO, Tokyo (JP)

(21) Appl. No.: 18/329,810

(22) Filed: Jun. 6, 2023

(30) Foreign Application Priority Data
Jun. 13, 2022 (JP) 2022-095224

(51) Int. Cl. A61B 6/00; G01T 1/17; G01N 23/046; H05G 1/46

(52) U.S. Cl. CPC A61B 6/482; G01T 1/17; G01N 2223/401; H05G 1/46; A61B 6/4241; G01N 23/046

(57) ABSTRACT

A radiation imaging apparatus includes: a processing unit configured to obtain a plurality of images corresponding to a plurality of different radiation energies by irradiating an object with radiation and performing imaging using an energy spectrum obtained by totaling energy information obtained by dividing a time-serially obtained radiation photon energy in a time direction, and perform energy subtraction processing using the plurality of images.

### BACKGROUND OF THE INVENTION

### Field of the Invention

The disclosed technique relates to a radiation imaging apparatus, an information processing apparatus, an information processing method, and a non-transitory computer-readable storage medium and, more particularly, to a radiation imaging apparatus used for still image capturing such as general imaging or moving image capturing such as fluoroscopic imaging in medical diagnosis, an information processing apparatus, an information processing method, and a non-transitory computer-readable storage medium.

### Description of the Related Art

A radiation imaging apparatus using a flat panel detector (to be abbreviated as an FPD hereinafter) formed by a semiconductor material is currently widespread as an imaging apparatus used for medical imaging diagnosis or non-destructive inspection by X-rays.

In energy subtraction processing that is an imaging method using an FPD, a plurality of images of different energies, which are obtained by emitting X-rays of different tube voltages, are processed, thereby obtaining a material decomposition image with a reduced contrast, for example, a bone image or a soft tissue image (International Publication No. 2019/181229).

However, since the time difference between a plurality of images is determined by the imaging time of the FPD, if the object moves during this time, for example, an artifact caused by the motion may be generated in the image obtained by energy subtraction processing.

Japanese Patent Laid-Open No. 2009-504221 describes a dual energy imaging system which generates X-ray pulses of different kV values on a time scale of millimeter second, samples and holds signal integration corresponding to a first sub-image at a first kV value, and performs signal integration corresponding to a second sub-image at a second kV value concurrently with readout of the first sub-image.

However, energy subtraction processing needs the information of a plurality of X-ray spectra of different radiation energies. For this reason, in an imaging method (to be also referred to as time division imaging hereinafter) for obtaining a plurality of sub-images by sampling signals with a very short time difference in an X-ray irradiation period of one pulse, it is difficult to measure the X-ray spectrum of each image, and processing using an X-ray spectrum estimated based on the pixel values of the sub-images is performed. This lowers the accuracy of subtraction processing.

The disclosed technique provides a technique capable of obtaining the energy spectrum of irradiated radiation.

### SUMMARY OF THE INVENTION

According to one aspect of the present invention, there is provided a radiation imaging apparatus comprising: a processing unit configured to obtain a plurality of images corresponding to a plurality of different radiation energies by irradiating an object with radiation and performing imaging using an energy spectrum obtained by totaling energy information obtained by dividing a time-serially obtained radiation photon energy in a time direction, and perform energy subtraction processing using the plurality of images.

Further features of the present invention will become apparent from the following description of exemplary embodiments (with reference to the attached drawings).

### BRIEF DESCRIPTION OF THE DRAWINGS

FIG. 1 is a view showing an example of the configuration of an X-ray imaging system according to a first embodiment;

FIG. 2 is an equivalent circuit diagram of a pixel in an X-ray imaging apparatus according to the first embodiment;

FIG. 3 is a timing chart of the X-ray imaging apparatus according to the first embodiment;

FIG. 4 is a timing chart of the X-ray imaging apparatus according to the first embodiment;

FIG. 5 is a view for explaining correction processing according to the first embodiment;

FIG. 6 is a block diagram of signal processing according to the first embodiment;

FIG. 7 is a block diagram of image processing according to the first embodiment;

FIGS. 8A to 8C are views for explaining the principle of time division of an X-ray spectrum according to the first embodiment;

FIG. 9 is a view showing the relationship between obtaining of an X-ray spectrum and the driving timing of the X-ray imaging system according to the first embodiment;

FIG. 10 is a flowchart showing the procedure of processing of the X-ray imaging system according to the first embodiment;

FIGS. 11A and 11B are timing charts for exemplarily explaining the waveform of X-rays;

FIG. 12 shows views for exemplarily explaining the principle of an image quality simulation;

FIG. 13 is a view exemplarily showing the correlation between the sample and hold timing and the quality of an image obtained by energy subtraction processing;

FIG. 14 is a flowchart showing the procedure of processing of an X-ray imaging system according to a second embodiment; and

FIG. 15 is a flowchart showing the procedure of processing of an X-ray imaging system according to a third embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Note that radiation according to the disclosed technique includes not only α-rays, β-rays, and γ-rays that are beams generated by particles (including photons) emitted by radioactive decay but also beams having equal or more energy, for example, X-rays, particle rays, and cosmic rays. In the following embodiments, an apparatus using X-rays as an example of radiation will be described. Therefore, an X-ray imaging apparatus and an X-ray imaging system will be described below as a radiation imaging apparatus and a radiation imaging system, respectively.

### FIRST EMBODIMENT

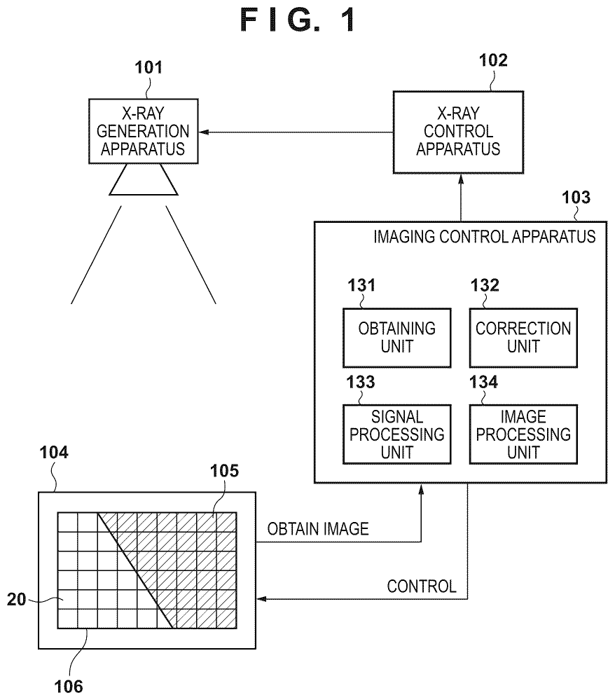

FIG. 1 is a block diagram showing an example of the configuration of an X-ray imaging system as an example of a radiation imaging system according to the first embodiment. The X-ray imaging system according to the first embodiment includes an X-ray generation apparatus 101, an X-ray control apparatus 102, an imaging control apparatus 103, and an X-ray imaging apparatus 104.

The X-ray generation apparatus 101 generates X-rays and irradiates an object with the X-rays. The X-ray control apparatus 102 controls generation of X-rays in the X-ray generation apparatus 101. The imaging control apparatus 103 includes, for example, one or a plurality of processors (CPUs) and a memory, and the processor executes a program stored in the memory to obtain an X-ray image and perform image processing. Note that each of processes including the image processing performed by the imaging control apparatus 103 may be implemented by dedicated hardware or by cooperation of hardware and software. The X-ray imaging apparatus 104 includes a phosphor 105 that converts X-rays into visible light, and a two-dimensional detector 106 that detects visible light. The two-dimensional detector is a sensor in which pixels 20 for detecting X-ray quanta are arranged in an array of X columns×Y rows, and outputs image information.

The imaging control apparatus 103 functions as an image processing apparatus that processes a radiation image by the above-described processor. An obtaining unit 131, a correction unit 132, a signal processing unit 133, and an image processing unit 134 indicate examples of the functional components of an information processing apparatus.

The obtaining unit 131 obtains a plurality of radiation images of energies different from each other, which are obtained by irradiating an object with radiation and performing imaging. The obtaining unit 131 obtains, as the plurality of radiation images, radiation images obtained by performing a sample and hold operation a plurality of times during one shot of radiation irradiation.

The correction unit 132 generates a plurality of images to be used for energy subtraction processing by correcting the plurality of radiation images obtained by the obtaining unit 131.

The signal processing unit 133 performs processing of obtaining an energy spectrum by totalizing energy information obtained by dividing a time-serially obtained X-ray photon energy in the time direction. The signal processing unit 133 counts the number of photons in time series in the energy information, thereby obtaining the energy spectrum. Also, the signal processing unit 133 obtains a plurality of images corresponding to a plurality of different radiation energies by irradiating an object with radiation and performing imaging using an energy spectrum obtained by totaling energy information obtained by dividing a time-serially obtained radiation photon energy in the time direction, and performs energy subtraction processing using the plurality of images. If the obtaining of the energy spectrum is applied to energy subtraction processing, the signal processing unit 133 performs energy subtraction processing using a plurality of images corresponding to a plurality of X-ray energies obtained by irradiating an object with X-rays and performing imaging and an energy spectrum obtained based on a time-serially obtained X-ray photon energy. Here, the signal processing unit 133 obtains the energy spectrum by totalizing the energy information obtained by dividing the X-ray photon energy at a timing of sampling and holding the signal of the X-ray energy.

In addition, the signal processing unit 133 generates a material characteristic image using a plurality of images generated by the correction unit 132. The material characteristic image is an image obtained in energy subtraction processing, such as a material decomposition image representing a decomposed material such as a bone or a soft tissue, or a material identification image representing an effective atomic number and its surface density. Based on a plurality of radiation images captured using different radiation energies, the signal processing unit 133 generates, for example, a first material decomposition image representing the thickness of a first material and a second material decomposition image representing the thickness of a second material. Also, the signal processing unit 133 generates a thickness image that combines the thickness of the first material and the thickness of the second material. Here, the first material includes at least calcium, hydoroxyapatite, or bone, and the second material includes at least water, fat, or a soft tissue that does not contain calcium. Details of the signal processing unit 133 will be described later. The image processing unit 134 generates a display image using the material characteristic image obtained by signal processing of the signal processing unit 133.

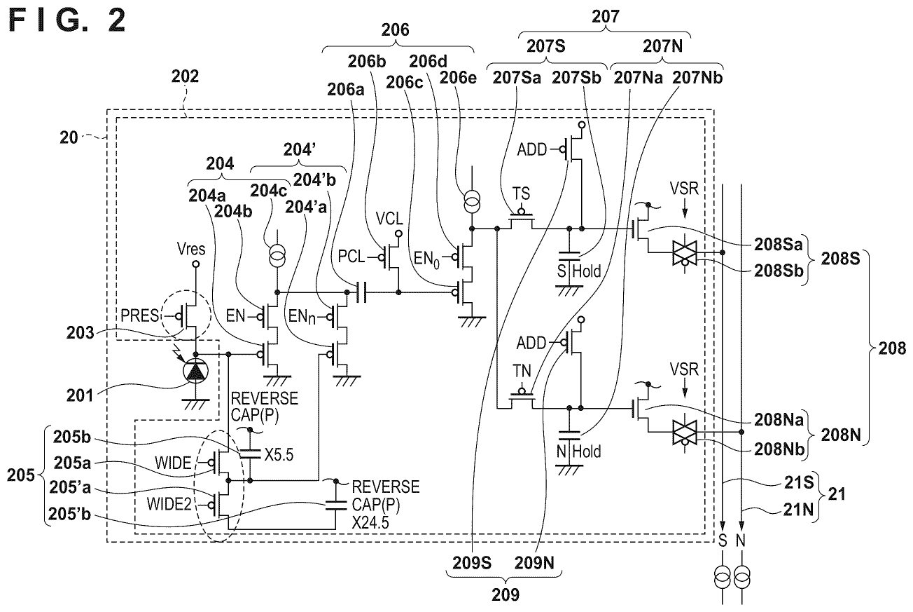

FIG. 2 is an equivalent circuit diagram of the pixel 20 according to the first embodiment. The pixel 20 includes a photoelectric converting element 201 and an output circuit unit 202. The photoelectric converting element 201 can typically be a photodiode. The output circuit unit 202 includes an amplification circuit unit 204, a clamp circuit unit 206, a sample and hold circuit unit 207, and a selection circuit unit 208.

The photoelectric converting element 201 includes a charge accumulation portion. The charge accumulation portion is connected to the gate of a MOS transistor 204a of the amplification circuit unit 204. The source of the MOS transistor 204a is connected to a current source 204c via a MOS transistor 204b. The MOS transistor 204a and the current source 204c form a source follower circuit. The MOS transistor 204b is an enable switch that is turned on when an enable signal EN supplied to its gate is set at an active level, and sets the source follower circuit in an operation state.

In the example shown in FIG. 2, the charge accumulation portion of the photoelectric converting element 201 and the gate of the MOS transistor 204a form a common node, and this node functions as a charge-voltage converter that converts charges accumulated in the charge accumulation portion into a voltage. That is, a voltage V (=Q/C) determined by charges Q accumulated in the charge accumulation portion and a capacitance value C of the charge-voltage converter appears in the charge-voltage converter. The charge-voltage converter is connected to a reset potential Vres via a reset switch 203. When a reset signal PRES is set at an active level, the reset switch 203 is turned on, and the potential of the charge-voltage converter is reset to the reset potential Vres.

The clamp circuit unit 206 clamps, by a clamp capacitor 206a, noise output from the amplification circuit unit 204 in accordance with the reset potential of the charge-voltage converter. That is, the clamp circuit unit 206 is a circuit configured to cancel the noise from a signal output from the source follower circuit in accordance with charges generated by photoelectric conversion in the photoelectric converting element 201. The noise includes kTC noise at the time of reset. Clamping is performed by turning on a MOS transistor 206b by setting a clamp signal PCL at an active level, and then turning off the MOS transistor 206b by setting the clamp signal PCL at an inactive level. The output side of the clamp capacitor 206a is connected to the gate of a MOS transistor 206c. The source of the MOS transistor 206c is connected to a current source 206e via a MOS transistor 206d. The MOS transistor 206c and the current source 206e form a source follower circuit. The MOS transistor 206d is an enable switch that is turned on when an enable signal ENO supplied to its gate is set at an active level, and sets the source follower circuit in an operation state. The signal output from the clamp circuit unit 206 in accordance with charges generated by photoelectric conversion in the photoelectric converting element 201 is written, as an optical signal, in a capacitor 207Sb via a switch 207Sa when an optical signal sampling signal TS is set at an active level. The signal output from the clamp circuit unit 206 when turning on the MOS transistor 206b immediately after resetting the potential of the charge-voltage converter is a clamp voltage. The noise signal is written in a capacitor 207Nb via a switch 207Na when a noise sampling signal TN is set at an active level. This noise signal includes an offset component of the clamp circuit unit 206. The switch 207Sa and the capacitor 207Sb form a signal sample and hold circuit 207S, and the switch 207Na and the capacitor 207Nb form a noise sample and hold circuit 207N. The sample and hold circuit unit 207 includes the signal sample and hold circuit 207S and the noise sample and hold circuit 207N.

When a driving circuit unit drives a row selection signal to an active level, the signal (optical signal) held in the capacitor 207Sb is output to a signal line 21S via a MOS transistor 208Sa and a row selection switch 208Sb. In addition, the signal (noise) held in the capacitor 207Nb is simultaneously output to a signal line 21N via a MOS transistor 208Na and a row selection switch 208Nb. The MOS transistor 208Sa forms a source follower circuit (not shown) with a constant current source provided on the signal line 21S. Similarly, the MOS transistor 208Na forms a source follower circuit (not shown) with a constant current source provided on the signal line 21N. The MOS transistor 208Sa and the row selection switch 208Sb form a signal selection circuit unit 208S, and the MOS transistor 208Na and the row selection switch 208Nb form a noise selection circuit unit 208N. The selection circuit unit 208 includes the signal selection circuit unit 208S and the noise selection circuit unit 208N.

The pixel 20 may include an addition switch 209S that adds the optical signals of the plurality of adjacent pixels 20. In an addition mode, an addition mode signal ADD is set at an active level, and the addition switch 209S is turned on. This causes the addition switch 209S to interconnect the capacitors 207Sb of the adjacent pixels 20, and the optical signals are averaged. Similarly, the pixel 20 may include an addition switch 209N that adds noise components of the plurality of adjacent pixels 20. When the addition switch 209N is turned on, the capacitors 207Nb of the adjacent pixels 20 are interconnected by the addition switch 209N, thereby averaging the noise components. An adder 209 includes the addition switches 209S and 209N.

Furthermore, the pixel 20 may include a sensitivity changing unit 205 for changing the sensitivity. The pixel 20 can include, for example, a first sensitivity change switch 205a, a second sensitivity change switch 205′a, and their circuit elements. When a first change signal WIDE is set at an active level, the first sensitivity change switch 205a is turned on to add the capacitance value of a first additional capacitor 205b to the capacitance value of the charge-voltage converter. This decreases the sensitivity of the pixel 20. When a second change signal WIDE2 is set at an active level, the second sensitivity change switch 205′a is turned on to add the capacitance value of a second additional capacitor 205′b to the capacitance value of the charge-voltage converter. This further decreases the sensitivity of the pixel 20. In this way, it is possible to receive a larger light amount by adding a function of decreasing the sensitivity of the pixel 20, thereby widening a dynamic range. When the first change signal WIDE is set at the active level, an enable signal ENw may be set at an active level to cause a MOS transistor 204′a to perform a source follower operation instead of the MOS transistor 204a.

The X-ray imaging apparatus 104 reads out the output of the above-described pixel circuit from the two-dimensional detector 106, causes an A/D converter (not shown) to covert the output into a digital value, and then transfers an image to the imaging control apparatus 103.

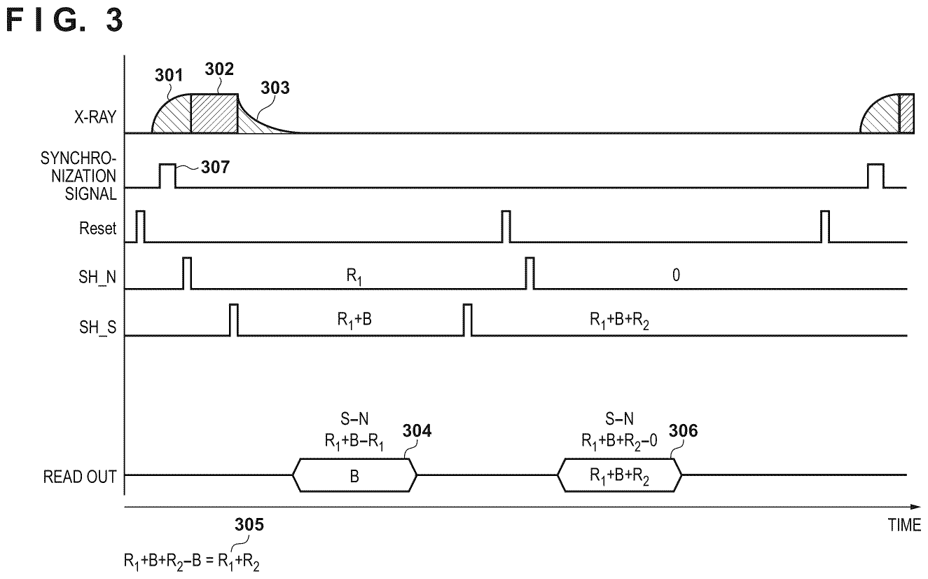

The operation of the X-ray imaging system having the above-described configuration according to the first embodiment will be described next. FIG. 3 shows the driving timing of the X-ray imaging apparatus 104 when energy subtraction is performed in the X-ray imaging system according to the first embodiment. When the abscissa represents the time, waveforms in FIG. 3 indicate timings of X-ray irradiation, a synchronization signal, reset of the photoelectric converting element 201, the sample and hold circuit 207, and readout of an image from a signal line 21.

After the reset signal resets the photoelectric converting element 201, X-ray irradiation is performed. The tube voltage of the X-rays ideally has a rectangular waveform but it takes a finite time for the tube voltage to rise or fall. Especially, if the time of irradiation of pulsed X-rays is short, the tube voltage is not considered to have a rectangular waveform any more, and has waveforms, as indicated by X-rays 301 to 303. The X-rays 301 during the rising period, the X-rays 302 during the stable period, and the X-rays 303 during the falling period have different X-ray energies. Therefore, by obtaining an X-ray image corresponding to radiation during a period divided by a sample and hold operation, a plurality of kinds of X-ray images of different energies are obtained.

The X-ray imaging apparatus 104 causes the noise sample and hold circuit 207N to perform sampling after irradiation of the X-rays 301 during the rising period, and causes the signal sample and hold circuit 207S to perform sampling after irradiation of the X-rays 302 during the stable period. After that, the X-ray imaging apparatus 104 reads out, as an image, the difference between the signal lines 21N and 21S. At this time, a signal (R₁) of the X-rays 301 during the rising period is held in the noise sample and hold circuit 207N, and the sum (R₁+B) of the signal of the X-rays 301 during the rising period and a signal (B) of the X-rays 302 during the stable period is held in the signal sample and hold circuit 207S. Therefore, an image 304 corresponding to the signal of the X-rays 302 during the stable period is read out.

Next, after completion of irradiation of the X-rays 303 during the falling period and readout of the image 304, the X-ray imaging apparatus 104 causes the signal sample and hold circuit 207S to perform sampling again. After that, the X-ray imaging apparatus 104 resets the photoelectric converting element 201, causes the noise sample and hold circuit 207N to perform sampling again, and reads out, as an image, the difference between the signal lines 21N and 21S. At this time, a signal in a state in which irradiation of X-rays is not performed is held in the noise sample and hold circuit 207N, and the sum (R₁+B+R₂) of the signal of the X-rays 301 during the rising period, the signal of the X-rays 302 during the stable period, and a signal (R₂) of the X-rays 303 during the falling period is held in the signal sample and hold circuit 207S. Therefore, an image 306 corresponding to the signal of the X-rays 301 during the rising period, the signal of the X-rays 302 during the stable period, and the signal of the X-rays 303 during the falling period is read out. After that, by calculating the difference between the images 306 and 304, an image 305 corresponding to the sum of the X-rays 301 during the rising period and the X-rays 303 during the falling period is obtained. This calculation processing may be performed by the X-ray imaging apparatus 104 or the imaging control apparatus 103.

The timing of resetting the sample and hold circuit 207 and the photoelectric converting element 201 is decided using a synchronization signal 307 indicating the start of irradiation of X-rays from the X-ray generation apparatus 101. As a method of detecting the start of irradiation of X-rays, a configuration for measuring the tube current of the X-ray generation apparatus 101 and determining whether the current value exceeds a preset threshold can be used but the present invention is not limited to this. For example, a configuration for detecting the start of application of X-rays by repeatedly reading out the pixel 20 and determining whether the pixel value exceeds a preset threshold after completion of the reset of the photoelectric converting element 201 may be used.

Alternatively, for example, a configuration for detecting the start of irradiation of X-rays by incorporating an X-ray detector different from the two-dimensional detector 106 in the X-ray imaging apparatus 104 and determining whether a measured value of the X-ray detector exceeds a preset threshold may be used. In either method, after a time designated in advance elapses after the input of the synchronization signal 307 indicating the start of application of X-rays, sampling of the signal sample and hold circuit 207S, sampling of the noise sample and hold circuit 207N, and reset of the photoelectric converting element 201 are performed.

As described above, the image 304 corresponding to the stable period of the pulsed X-rays and the image 305 corresponding to the sum of the signal during the rising period and that during the falling period are obtained. Since the energies of the X-rays irradiated when forming the two X-ray images are different, calculation is performed for the X-ray images, thereby making it possible to perform energy subtraction processing.

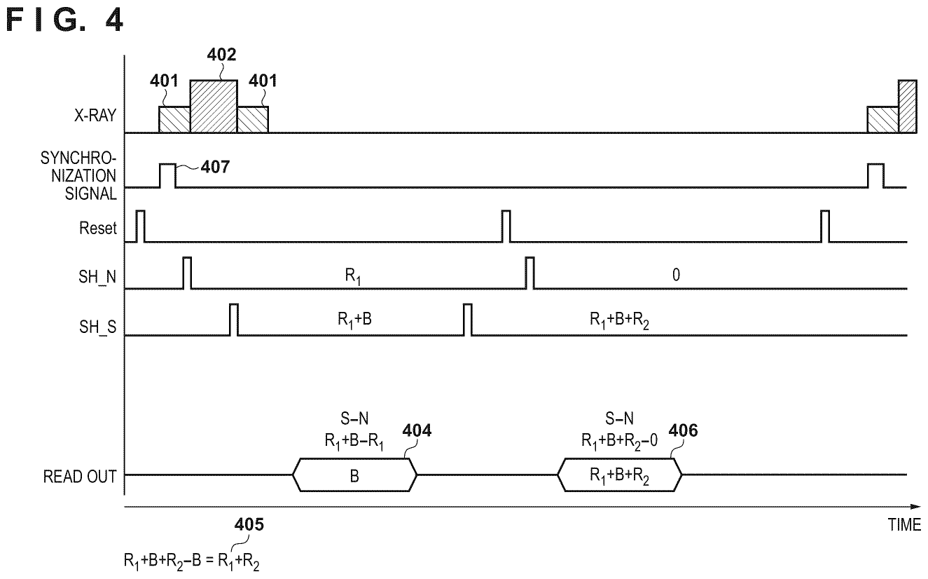

FIG. 4 shows the driving timing of the X-ray imaging apparatus 104 when energy subtraction is performed in the X-ray imaging system according to the first embodiment. The driving timing shown in FIG. 4 is different from the driving timing shown in FIG. 3 in that the tube voltage of the X-ray generation apparatus 101 is actively switched.

First, after the reset of the photoelectric converting element 201, the X-ray generation apparatus 101 performs irradiation of low energy X-rays 401. In this state, the X-ray imaging apparatus 104 causes the noise sample and hold circuit 207N to perform sampling. After that, the X-ray generation apparatus 101 switches the tube voltage to perform irradiation of high energy X-rays 402. In this state, the X-ray imaging apparatus 104 causes the signal sample and hold circuit 207S to perform sampling. After that, the X-ray generation apparatus 101 switches the tube voltage to perform irradiation of low energy X-rays 403. The X-ray imaging apparatus 104 reads out, as an image, the difference between the signal lines 21N and 21S. At this time, a signal (R₁) of the low energy X-rays 401 is held in the noise sample and hold circuit 207N, and the sum (R₁+B) of the signal of the low energy X-rays 401 and a signal (B) of the high energy X-rays 402 is held in the signal sample and hold circuit 207S. Therefore, an image 404 corresponding to the signal of the high energy X-rays 402 is read out.

Next, after completion of the irradiation of the low energy X-rays 403 and the readout of the image 404, the X-ray imaging apparatus 104 causes the signal sample and hold circuit 207S to perform sampling again. After that, the X-ray imaging apparatus 104 resets the photoelectric converting element 201, causes the noise sample and hold circuit 207N to perform sampling again, and reads out, as an image, the difference between the signal lines 21N and 21S. At this time, a signal in a state in which no X-rays are applied is held in the noise sample and hold circuit 207N, and the sum (R₁+B+R₂) of the signal of the low energy X-rays 401, the signal of the high energy X-rays 402, and a signal (R₂) of the low energy X-rays 403 is held in the signal sample and hold circuit 207S. Therefore, an image 406 corresponding to the signal of the low energy X-rays 401, the signal of the high energy X-rays 402, and the signal of the low energy X-rays 403 is read out.

After that, by calculating the difference between the images 406 and 404, an image 405 corresponding to the sum of the low energy X-rays 401 and the low energy X-rays 403 is obtained. This calculation processing may be performed by the X-ray imaging apparatus 104 or the imaging control apparatus 103. With respect to a synchronization signal 407, the same as in FIG. 3 applies. As described above, by obtaining images while actively switching the tube voltage, the energy difference between radiation images of low energy and high energy can be made large, as compared with the method shown in FIG. 3.

Next, energy subtraction processing by the imaging control apparatus 103 will be described. The energy subtraction processing according to the first embodiment is divided into three stages of correction processing by the correction unit 132, signal processing by the signal processing unit 133, and image processing by the image processing unit 134. Each process will be described below.

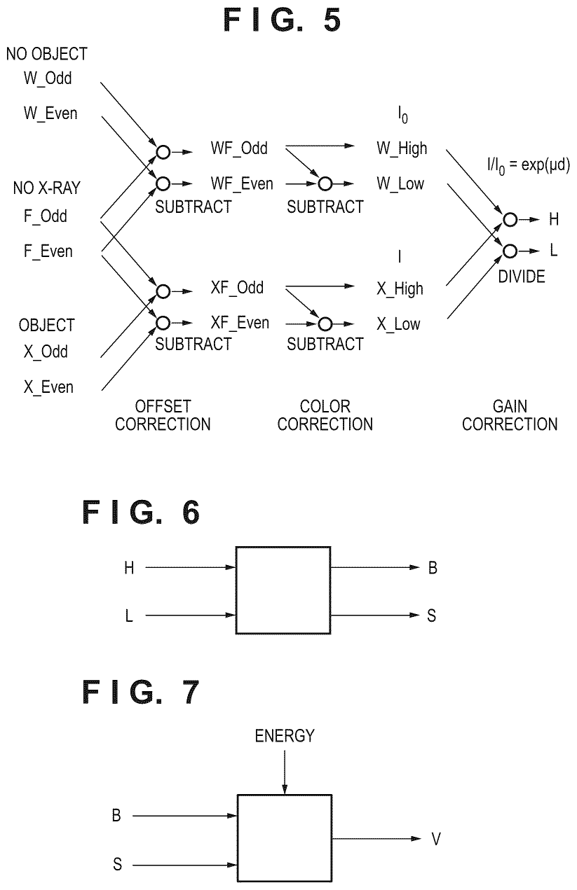

The correction processing is processing of generating, by processing a plurality of radiation images obtained from the X-ray imaging apparatus 104, a plurality of images to be used for the signal processing (to be described later) in the energy subtraction processing. FIG. 5 is a block diagram of the correction processing for the energy subtraction processing according to the first embodiment. First, the obtaining unit 131 causes the X-ray imaging apparatus 104 to perform imaging in a state in which irradiation of X-rays is not performed, thereby obtaining images by the driving operation shown in FIG. 3 or 4. With this driving operation, two images are read out. The first image (image 304 or 404) will be referred to as F_ODD hereinafter and the second image (image 306 or 406) will be referred to as F_EVEN hereinafter. Each of F_ODD and F_EVEN is an image corresponding to FPN (Fixed Pattern Noise) of the X-ray imaging apparatus 104.

Next, the obtaining unit 131 causes the X-ray imaging apparatus 104 to perform imaging by performing irradiation of X-rays in a state in which there is no object, thereby obtaining gain correction images output from the X-ray imaging apparatus 104 by the driving operation shown in FIG. 3 or 4. With this driving operation, two images are read out, similar to the above operation. The first gain correction image (image 304 or 404) will be referred to as W_ODD hereinafter and the second gain correction image (image 306 or 406) will be referred to as W_EVEN hereinafter. Each of W_ODD and W_EVEN is an image corresponding to the sum of the FPN of the X-ray imaging apparatus 104 and the signal by X-rays. The correction unit 132 subtracts F_ODD from W_ODD and F_EVEN from W_EVEN, thereby obtaining images WF_ODD and WF_EVEN from each of which the FPN of the X-ray imaging apparatus 104 has been removed. This is called offset correction.

WF_ODD is an image corresponding to the X-rays 302 during the stable period, and WF_EVEN is an image corresponding to the sum of the X-rays 301 during the rising period, the X-rays 302 during the stable period, and the X-rays 303 during the falling period. Therefore, the correction unit 132 obtains an image corresponding to the sum of the X-rays 301 during the rising period and the X-rays 303 during the falling period by subtracting WF_ODD from WF_EVEN. The processing of obtaining an image corresponding to X-rays during a specific period divided by the sample and hold operation by subtraction of a plurality of images is called color correction. The energy of the X-rays 301 during the rising period and that of the X-rays 303 during the falling period are lower than the energy of the X-rays 302 during the stable period. Therefore, by subtracting WF_ODD from WF_EVEN by color correction, a low energy image W_Low when there is no object is obtained. Furthermore, a high energy image W_High when there is no object is obtained from WF_ODD.

Next, the obtaining unit 131 causes the X-ray imaging apparatus 104 to perform imaging by performing irradiation of X-rays in a state in which there is an object, thereby obtaining images output from the X-ray imaging apparatus 104 by the driving operation shown in FIG. 3 or 4. At this time, two images are read out. The first image (image 304 or 404) will be referred to as X_ODD hereinafter and the second image (image 306 or 406) will be referred to as X_EVEN hereinafter. The correction unit 132 performs the same offset correction processing and color correction processing as those when there is no object, thereby obtaining a low energy image X_Low when there is the object and a high energy image X_High when there is the object.

When d represents the thickness of the object, μ represents the linear attenuation coefficient of the object, I₀ represents the output of the pixel 20 when there is no object, and I represents the output of the pixel 20 when there is the object, equation (1) below holds.

I=I₀ exp(μd) (1)

Equation (1) is modified to obtain equation (2) below. The right-hand side of equation (2) represents the attenuation rate of the object. The attenuation rate of the object is a real number between 0 and 1.

I/I₀=exp(μd) (2)

Therefore, the correction unit 132 obtains the attenuation rate image L at low energy (to be also referred to as the “low energy image L” hereinafter) by dividing the low energy image X_Low when there is the object by the low energy image W_Low when there is no object. Similarly, the correction unit 132 obtains the attenuation rate image H at high energy (to be also referred to as the “high energy image H” hereinafter) by dividing the high energy image X_High when there is the object by the high energy image W_High when there is no object. The processing of obtaining an image (L or H) of an attenuation rate at low energy or an attenuation rate at high energy by dividing an image obtained based on a radiation image obtained when there is an object by an image obtained based on a radiation image obtained when there is no object is called gain correction.

FIG. 6 is a block diagram of the signal processing of the energy subtraction processing according to the first embodiment. The signal processing unit 133 generates a material characteristic image using a plurality of images obtained from the correction unit 132. Generation processing of a material decomposition image formed from a bone thickness image B (to be also referred to as a bone image B hereinafter) and a soft tissue thickness image S (to be also referred to as a soft tissue image S hereinafter) will be described below. The signal processing unit 133 performs the following processing to obtain the bone thickness image B and the soft tissue thickness image S from the attenuation rate image L at low energy and the attenuation rate image H at high energy, both of which have been obtained by the correction processing shown in FIG. 5.

First, when E represents the energy of X-ray photons, N(E) represents the number of photons at the energy E, B represents a thickness in a bone thickness image, S represents a thickness in a soft tissue thickness image, μ_{B}(E) represents the linear attenuation coefficient of the bone at the energy E, μ_{S}(E) represents the linear attenuation coefficient of the soft tissue at the energy E, and I/I₀ represents the attenuation rate, equation (3) below holds.

$$I / {I}_{0} = \frac{{\int }_{0}^{∞} N ( E ) exp \{ - {μ}_{B} ( E ) B - {μ}_{S} ( E ) S \} EdE}{{\int }_{0}^{∞} N ( E ) EdE} \qquad ( 3 )$$

The number N(E) of photons at the energy E is an X-ray spectrum. The X-ray spectrum is obtained by simulation or actual measurement. Each of the linear attenuation coefficient μ_{B}(E) of the bone at the energy E and the linear attenuation coefficient μ_{S}(E) of the soft tissue at the energy E is obtained from a database of NIST (National Institute of Standards and Technology) or the like. Therefore, according to equation (3), it is possible to calculate the attenuation rate I/I₀ for the thickness B in an arbitrary bone thickness image, the thickness S in a soft tissue thickness image, and the X-ray spectrum N(E).

When N_{L}(E) represents a low energy X-ray spectrum and N_{H}(E) represents a high energy X-ray spectrum, equations (4) below hold concerning the attenuation rate of the image L and the attenuation rate of the image H. Note that in the following explanation, the attenuation rate of the image L shown in equations (4) will also simply be referred to as the attenuation rate L at low energy, and the attenuation rate of the image H will also simply be referred to as the attenuation rate H at high energy.

$$\begin{matrix}L = \frac{{\int }_{0}^{∞} {N}_{L} ( E ) exp \{ - {μ}_{B} ( E ) B - {μ}_{S} ( E ) S \} EdE}{{\int }_{0}^{∞} {N}_{L} ( E ) EdE} \\ H = \frac{{\int }_{0}^{∞} {N}_{H} ( E ) exp \{ - {μ}_{B} ( E ) B - {μ}_{S} ( E ) S \} EdE}{{\int }_{0}^{∞} {N}_{H} ( E ) EdE}\end{matrix} \qquad ( 4 )$$

By solving nonlinear simultaneous equations (4), the thickness B in the bone thickness image and the thickness S in the soft tissue thickness image are obtained. A case in which the Newton-Raphson method is used as a representative method of solving the nonlinear simultaneous equations will be explained. When m represents an iteration count of the Newton-Raphson method, B^{m} represents a bone thickness after the mth iteration, and S^{m} represents a soft tissue thickness after the mth iteration, an attenuation rate H^{m} at high energy after the mth iteration and an attenuation rate Lm at low energy after the mth iteration are given by:

$$\begin{matrix}{L}^{m} = \frac{{\int }_{0}^{∞} {N}_{L} ( E ) exp \{ - {μ}_{B} ( E ) B - {μ}_{S} ( E ) {S}^{m} \} EdE}{{\int }_{0}^{∞} {N}_{L} ( E ) EdE} \\ {H}^{m} = \frac{{\int }_{0}^{∞} {N}_{H} ( E ) exp \{ - {μ}_{B} ( E ) B - {μ}_{S} ( E ) {S}^{m} \} EdE}{{\int }_{0}^{∞} {N}_{H} ( E ) EdE}\end{matrix} \qquad ( 5 )$$

The change rates of the attenuation rates when the thicknesses slightly change are given by:

$$\begin{matrix}\frac{\partial {H}^{m}}{\partial {B}^{m}} = \frac{{\int }_{0}^{∞} - {μ}_{B} ( E ) {N}_{H} ( E ) exp \{ - {μ}_{B} ( E ) {B}^{m} - {μ}_{S} ( E ) {S}^{m} \} EdE}{{\int }_{0}^{∞} {N}_{H} ( E ) EdE} \\ \frac{\partial {L}^{m}}{\partial {B}^{m}} = \frac{{\int }_{0}^{∞} - {μ}_{B} ( E ) {N}_{L} ( E ) exp \{ - {μ}_{B} ( E ) {B}^{m} - {μ}_{S} ( E ) {S}^{m} \} EdE}{{\int }_{0}^{∞} {N}_{L} ( E ) EdE} \\ \frac{\partial {H}^{m}}{\partial {S}^{m}} = \frac{{\int }_{0}^{∞} - {μ}_{S} ( E ) {N}_{H} ( E ) exp \{ - {μ}_{B} ( E ) {B}^{m} - {μ}_{S} ( E ) {S}^{m} \} EdE}{{\int }_{0}^{∞} {N}_{H} ( E ) EdE} \\ \frac{\partial {L}^{m}}{\partial {S}^{m}} = \frac{{\int }_{0}^{∞} - {μ}_{S} ( E ) {N}_{L} ( E ) exp \{ - {μ}_{B} ( E ) {B}^{m} - {μ}_{S} ( E ) {S}^{m} \} EdE}{{\int }_{0}^{∞} {N}_{L} ( E ) EdE}\end{matrix} \qquad ( 6 )$$

At this time, using the attenuation rate H at high energy and the attenuation rate L at low energy, a bone thickness B^{m+1} and a soft tissue thickness S^{m+1} after the (m+1)th iteration are given by:

$$[ \begin{matrix}{B}^{m + 1} \\ {S}^{m + 1}\end{matrix} ] = [ \begin{matrix}{B}^{m} \\ {S}^{m}\end{matrix} ] + {[ \begin{matrix}\frac{\partial {H}^{m}}{\partial {B}^{m}} & \frac{\partial {H}^{m}}{\partial {S}^{m}} \\ \frac{\partial {L}^{m}}{\partial {B}^{m}} & \frac{\partial {L}^{m}}{\partial {S}^{m}}\end{matrix} ]}^{- 1} [ \begin{matrix}H - {H}^{m} \\ L - {L}^{m}\end{matrix} ] \qquad ( 7 )$$

When det represents a determinant, the inverse matrix of a 2×2 matrix is given, using the Cramer's rule, by:

$$det = \frac{\partial {H}^{m}}{\partial {B}^{m}} \frac{\partial {L}^{m}}{\partial {S}^{m}} - \frac{\partial {H}^{m}}{\partial {S}^{m}} \frac{\partial {L}^{m}}{\partial {B}^{m}} \qquad ( 8 )$$

$${[ \begin{matrix}\frac{\partial {H}^{m}}{\partial {B}^{m}} & \frac{\partial {H}^{m}}{\partial {S}^{m}} \\ \frac{\partial {L}^{m}}{\partial {B}^{m}} & \frac{\partial {L}^{m}}{\partial {S}^{m}}\end{matrix} ]}^{- 1} = \frac{1}{det} [ \begin{matrix}\frac{\partial {L}^{m}}{\partial {S}^{m}} & - \frac{\partial {H}^{m}}{\partial {S}^{m}} \\ - \frac{\partial {L}^{m}}{\partial {B}^{m}} & \frac{\partial {H}^{m}}{\partial {B}^{m}}\end{matrix} ]$$

Therefore, by substituting equation (8) into equation (7), equations (9) below are obtained.

$$\begin{matrix}{B}^{m + 1} = {B}^{m} + \frac{1}{det} \frac{\partial {L}^{m}}{\partial {S}^{m}} ( H - {H}^{m} ) - \frac{1}{det} \frac{\partial {H}^{m}}{\partial {S}^{m}} ( L - {L}^{m} ) \\ {S}^{m + 1} = {S}^{m} - \frac{1}{det} \frac{\partial {L}^{m}}{\partial {B}^{m}} ( H - {H}^{m} ) + \frac{1}{det} \frac{\partial {H}^{m}}{\partial {B}^{m}} ( L - {L}^{m} )\end{matrix} \qquad ( 9 )$$

When the above calculation processing is repeated, the difference between the attenuation rate H^{m} at high energy after the mth iteration and the actually measured attenuation rate H at high energy approaches almost 0. The same applies to the attenuation rate L at low energy. This causes the bone thickness B^{m} after the mth iteration to converge to the bone thickness B, and causes the soft tissue thickness S^{m} after the mth iteration to converge to the soft tissue thickness S. As described above, the nonlinear simultaneous equations (4) can be solved. Therefore, by calculating equations (4) for all the pixels, the bone thickness image B and the soft tissue thickness image S can be obtained from the attenuation rate image L at low energy and the attenuation rate image H at high energy.

Note that the bone thickness image B and the soft tissue thickness image S are calculated in the first embodiment but the disclosed technique is not limited to this. For example, a water thickness W and a contrast agent thickness I may be calculated. That is, decomposition may be performed into the thicknesses of arbitrary two kinds of materials. In addition, an image of an effective atomic number Z and an image of a surface density D may be obtained from the attenuation rate image L at low energy and the attenuation rate image H at high energy, which are obtained by the correction shown in FIG. 5. The effective atomic number Z is an equivalent atomic number of a mixture, and the surface density D is the product of the density [g/cm³] of an object and the thickness [cm] of the object.

Also, in the first embodiment, the nonlinear simultaneous equations are solved using the Newton-Raphson method. However, the disclosed technique is not limited to this. For example, an iterative method such as a least square method or a bisection method may be used. Furthermore, in the first embodiment, the nonlinear simultaneous equations are solved using the iterative method but the disclosed technique is not limited to this. A configuration for generating a table by obtaining, in advance, the bone thicknesses B and the soft tissue thicknesses S for various combinations of the attenuation rates H at high energy and the attenuation rates L at low energy, and obtaining the bone thickness B and the soft tissue thickness S at high speed by referring to this table may be used.

FIG. 7 is a block diagram of image processing of energy subtraction processing according to the first embodiment. The image processing unit 134 according to the first embodiment performs image processing of obtaining a virtual monochromatic X-ray image from the bone thickness image B and the soft tissue thickness image S obtained by the signal processing shown in FIG. 6. The virtual monochromatic X-ray image is an image assumed to be obtained by irradiation of X-rays of a single energy. For example, letting E_{V} be the energy of virtual monochromatic X-rays, the virtual monochromatic X-ray image V is obtained by

V=exp{−μB(EV)B−μ_{S}(EV)S} (10)

The virtual monochromatic X-ray image is used in Dual Energy CT that combines energy subtraction and three-dimensional reconstruction. At this time, to improve the Contrast-To-Noise Ratio (CNR) of the virtual monochromatic X-ray image, the energy EV of virtual monochromatic X-rays is changed. For example, the linear attenuation coefficient μ_{B}(E) of a bone is larger than the linear attenuation coefficient μ_{S}(E) of a soft tissue. However, the larger the energy E_{V} of virtual monochromatic X-rays is, the smaller the difference between μ_{B}(E) and μ_{S}(E) is. Hence, an increase of noise in the virtual monochromatic X-ray image due to noise in the bone image is suppressed. On the other hand, the smaller the energy E_{V} of virtual monochromatic X-rays is, the larger the difference between μ_{B}(E) and μ_{S}(E) is. That is, an appropriate value exists for the energy E_{V} of the virtual monochromatic X-ray image.

Note that in this embodiment, the virtual monochromatic X-ray image is generated from the bone thickness B and the soft tissue thickness S. However, the present invention is not limited to this form. As described above, after the effective atomic number Z and the surface density D are calculated, the virtual monochromatic X-ray image may be generated using the effective atomic number Z and the surface density D. In addition, a combined X-ray image may be generated by combining a plurality of virtual monochromatic X-ray images generated using a plurality of energies E v. The combined X-ray image is an image assumed to be obtained by irradiation of X-rays of an arbitrary spectrum.

In image processing according to this embodiment, a virtual monochromatic X-ray image is generated. However, the present invention is not limited to this form. The bone thickness image B or the soft tissue thickness image S may directly be displayed. Alternatively, an image obtained by applying a filter in the time direction such as a recursive filter or a filter in the special direction such as a Gaussian filter to the bone thickness image B or the soft tissue thickness image S may be displayed. A Digital Subtraction Angiography (DSA) image of a bone may be obtained using a low energy image (attenuation rate) and a high energy image (attenuation rate) before and after injection of a contrast agent, and the DSA image may be displayed. That is, it can be said that image processing according to this embodiment is processing of performing an arbitrary operation for an image after image processing.

Note that the DSA image is obtained by, for example, the following method. First, before injection of a contrast agent, X-ray imaging is performed, thereby obtaining an attenuation rate image LM at low energy and an attenuation rate image H_{M} at high energy. A mask image B_{M} of the bone thickness and a mask image S_{M} of the soft tissue thickness are obtained from the image L_{M} and the image H_{M}. Next, a live image B_{L} of the bone thickness and a live image S_{L} of a soft tissue thickness are obtained from an attenuation rate image L_{L} at low energy and an attenuation rate image H_{L} at high energy, which are captured after the injection of the contrast agent. When the mask image B_{M} of the bone thickness is subtracted from the live image B_{L} of the bone thickness, a DSA image B_{DSA} of the bone is obtained.

The energy subtraction processing according to this embodiment is formed by three steps, correction processing, signal processing, and image processing, as shown in FIGS. 5 to 7. At this time, the bone thickness B and the soft tissue thickness S obtained by solving equations (4) are each defined as an estimated value of thickness. Also, a thickness measured by a measuring device or the like is defined as a true value of thickness. If correction processing and signal processing are appropriately performed, the estimated value and the true value of thickness must match. However, the present inventor made examinations and found that the estimated value of thickness obtained by the above-described energy subtraction processing done not necessarily match the true value of thickness. If the error between the estimated value of thickness and the true value of thickness is large, an artifact occurs in the image after image processing.

As a result of examinations made by the present inventor, it was found that causes of an error included scattered rays, the dose dependence of the attenuation rate, the attenuation rate thickness, and the X-ray spectrum. In this embodiment, a method of reducing an error caused by the X-ray spectrum is proposed.

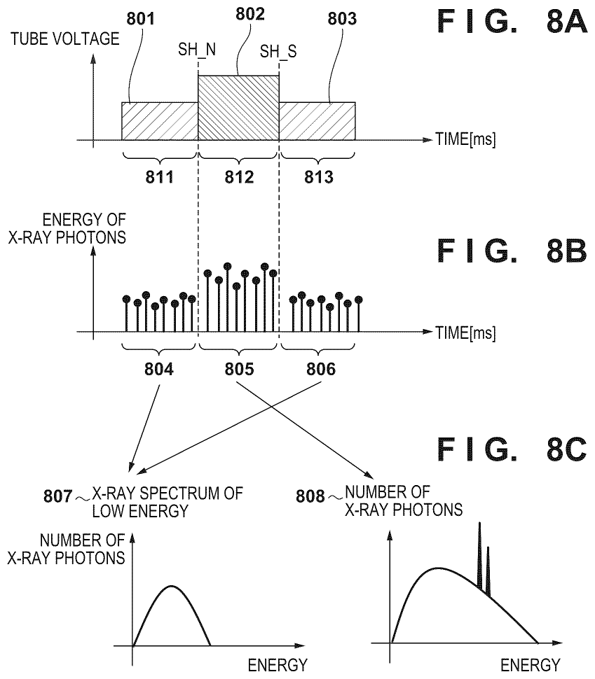

FIGS. 8A to 8C are views for explaining the principle of time division of an X-ray spectrum according to this embodiment. Processing of time-serially obtaining an X-ray photon energy (radiation photon energy) and dividing the obtained X-ray photon energy (radiation photon energy) in the time direction will be referred to as time division of an X-ray spectrum. Also, the X-ray photon energy divided in the time direction will be referred to as energy information. Here, as an example, the waveform of convex X-ray pulses is used. The X-ray generation apparatus 101 (radiation generation apparatus) switches the tube voltage and generates radiation. When generating convex X-ray pulses, the X-ray generation apparatus 101 (radiation generation apparatus) switches between a first tube voltage, a second tube voltage higher than the first tube voltage, and the first tube voltage, thereby generating X-ray pulses (convex X-ray pulses).

FIG. 8A is a view showing the relationship between time and the tube voltage. In FIG. 8A, reference numerals 801 to 803 indicate X-ray waveforms for which time is plotted along the abscissa, and the tube voltage is plotted along the ordinate. A section 811 indicates a low energy section, a section 812 indicates a high energy section, and a section 813 indicates a low energy section.

FIG. 8B is a view showing the relationship between time and the X-ray photon energy. In FIG. 8B, reference numerals 804 to 806 each denote the energy information of each X-ray photon for which time is plotted along the abscissa, and the X-ray photon energy is plotted along the ordinate. The signal processing unit 133 divides the X-ray photon energy at a timing (for example, SH_N) of sampling and holding a signal corresponding to first radiation energy (low energy) and at a timing (for example, SH_S) of sampling and holding a signal corresponding to second radiation energy higher than the first radiation energy.

Reference numerals 804 and 806 denote X-ray photon groups (X-ray photon groups of low energy) in the low energy sections 811 and 813 of the convex X-ray pulses. Similarly, reference numeral 805 denotes an X-ray photon group (an X-ray photon group of high energy) in the high energy section 812 of the convex X-ray pulses.

FIG. 8C is a view showing the relationship between the X-ray energy and the number of photons. Pieces of energy information of X-ray photons in the X-ray photon groups 804 and 806 are totalized. When the abscissa represents the X-ray energy, and the ordinate represents the number of photons, an X-ray spectrum 807 of low energy as shown in FIG. 8C can be obtained. Similarly, pieces of energy information of X-ray photons in the X-ray photon group 805 are totalized. When the abscissa represents the X-ray energy, and the ordinate represents the number of photons, an X-ray spectrum 808 of high energy can be obtained. The X-ray spectrum 807 has a shape according to the X-ray energies (801 and 803) in the totalization sections (811 and 813). The X-ray spectrum 808 has a shape according to the X-ray energy (802) in the totalization section (812).

Thus, when the data of time X-ray photon energy (time series X-ray photon energy) is time-divided and totalized, the X-ray spectra 807 and 808 that temporally change in one pulse can be obtained.

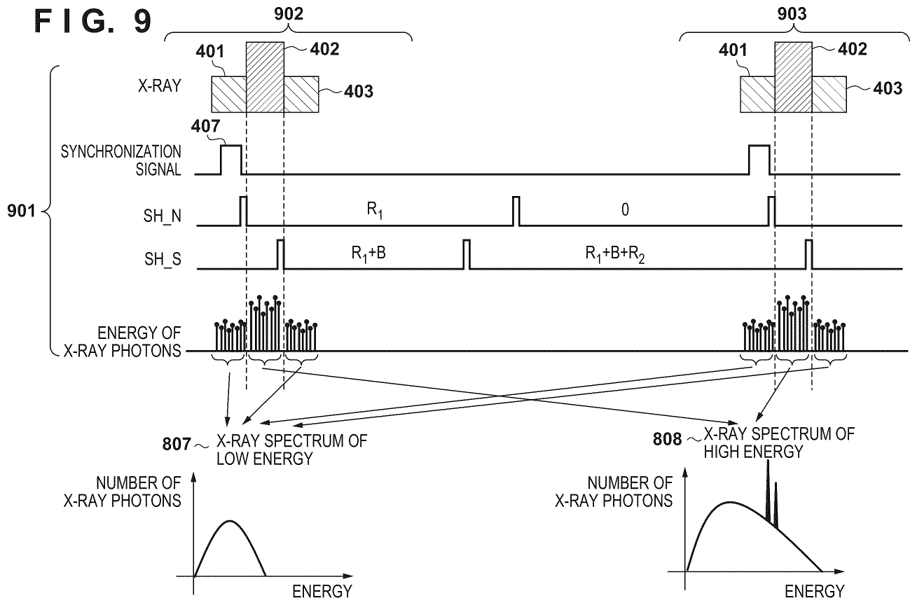

FIG. 9 shows the relationship between obtaining of an X-ray spectrum and the driving timing of the X-ray imaging system according to this embodiment. Driving of the X-ray imaging system has been described above with reference to FIG. 4. In FIG. 9, to avoid a repetitive description, reset and the read timing shown in FIG. 4 are not illustrated. Also, a description of reference numerals common to FIG. 4 will be omitted.

In FIG. 9, 901 shows the relationship between X-rays, a synchronization signal, the sample and hold timings SH_N and SH_S, and the X-ray photon energy (time X-ray photon energy), for which time is plotted along the abscissa. Also, as shown in FIG. 8C, reference numerals 807 and 808 show the distributions of X-ray spectra for which the X-ray energy is plotted along the abscissa, and the number of photons of X-rays is plotted along the ordinate. Reference numeral 807 indicates the distribution of the X-ray spectrum of low energy; and 808, the distribution of the X-ray spectrum of high energy.

During the X-ray irradiation period, a sample and hold operation according to the synchronization signal and measurement of the energy (time X-ray photon energy) of each X-ray photon are performed. Time division of the obtained time X-ray photon energy is performed such that the timing matches each of the sample and hold operations SH_N and SH_S in the driving of the X-ray imaging apparatus 104, as indicated by the dotted lines. Thus, the X-ray energy of the image (sub-image) obtained by the sample and hold operation matches the X-ray energy of the time-divided X-ray spectrum. That is, the measured X-ray spectra of a plurality of images (sub-images (H and L)) of different X-ray energies are obtained. Here, as shown in FIG. 4, the image 405 corresponding to the sum of the X-rays 401 of low energy and the X-rays 403 of low energy is defined as the sub-image 405. Also, the image 406 corresponding to the signal of the X-rays 401 of low energy, the signal of the X-rays 402 of high energy, and the signal of the X-rays 403 of low energy is defined as the sub-image 406.

Here, the image 405 (sub-image 405) corresponds to the attenuation rate image L at low energy in equations (4), and the image 406 (sub-image 406) corresponds to the attenuation rate image H at high energy. The X-ray spectrum 807 of low energy shown in FIGS. 8C and 9 corresponds to the spectrum N_{L}(E) of low energy X-rays in equations (4). Similarly, the X-ray spectrum 808 of high energy corresponds to the spectrum N_{H}(E) of high energy X-rays in equations (4). The linear attenuation coefficient μ_{B}(E) of the bone at the energy E and the linear attenuation coefficient μ_{S}(E) of the soft tissue at the energy E are obtained from a database of NIST or the like.

When simultaneous equations (4) are solved using the sub-image 405 and the X-ray spectrum 807, and the sub-image 406 and the X-ray spectrum 808, an accurate energy subtraction image can be obtained. For example, the bone thickness image B and the soft tissue thickness image S which are accurate can be obtained as material decomposition images.

Note that to measure the time X-ray photon energy, for example, an X-ray photon detector using cadmium telluride (CdTe) or the like can be used. Alternatively, an X-ray spectrometer incorporating a CdTe detector can be used. Normally, the X-ray spectrometer automatically totalizes the energy information of X-ray photons and outputs an X-ray spectrum. The X-ray spectrometer can obtain the data of time X-ray photon energy by obtaining stored data before totalization. In this embodiment, the X-ray photon detector or the X-ray spectrometer can function as an obtaining unit configured to time-serially obtain X-ray photon energy (radiation photon energy).

To increase the matching between the timing of time division of the X-ray photon group and the sample and hold timing of the X-ray imaging apparatus 104, the signal processing unit 133 performs signal processing for establishing synchronization between the X-ray generation apparatus 101 and the X-ray imaging apparatus 104 and the X-ray photon detector. For example, the synchronization signal 407 used to establish synchronization between the X-ray generation apparatus 101 and the X-ray imaging apparatus 104 may be used for synchronization of X-ray photon detection. By the signal processing of the signal processing unit 133, the X-ray photon detector obtains X-ray photon energy (radiation photon energy) in synchronism with radiation irradiation based on the temporally changing tube voltage.

When obtaining the X-ray spectrum, the intensity of X-rays can be limited by conditions such as the tube voltage, the tube current, and the irradiation time of the X-ray generation apparatus 101 or constraints by pile-up of the X-ray photon detector. The number of energy information of X-ray photons obtained by one pulse of X-rays is several ten to several hundred. To obtain a more accurate X-ray spectrum, the number of energy information is preferably increased.

Hence, if X-ray irradiation is performed a plurality of times, the signal processing unit 133 totalizes, for each X-ray energy, X-ray photon energies divided at the timing of sampling and holding the signal of X-ray energy, thereby obtaining energy information.

The X-ray photon detector can time-serially obtain X-ray photon energies when X-ray irradiation is performed at least once or a plurality of times. If X-ray irradiation is performed a plurality of times, the signal processing unit 133 totalizes, for each radiation energy, X-ray photon energies divided at the timing of sampling and holding the signal of X-ray energy, thereby obtaining energy information.

For example, as indicated by 902 and 903 in FIG. 9, the X-ray spectra 807 and 808 may be generated by totalizing pieces of information of X-ray pulses of a plurality of times for each energy. At this time, the timing (division timing) of time division of the X-ray pulses needs to match in each irradiation. Hence, if X-ray irradiation is performed a plurality of times, the signal processing unit 133 divides the X-ray photon energy at a timing matching each irradiation. For example, the timing of time division in each irradiation is preferably matched by resetting the timer of the X-ray photon detector in each X-ray irradiation. If the shape of the X-ray spectrum after totalization is rough because of a shortage of energy information of X-ray photons even if the pieces of information of X-ray pulses of the plurality of times are totalized for each energy, the shape may be corrected to a smooth shape by smoothing correction.

A plurality of X-ray spectra obtained by the above-described X-ray spectrum obtaining method are measured values, and can include wrong energy information of X-ray photons due to pile-up of the X-ray photon detector. For this reason, the information is preferably used for the operation of energy subtraction after only the wrong information of X-ray photons is removed by pile-up correction. The pile-up correction at this time is preferably performed for each spectrum after time division. If the X-ray spectrum (a low energy spectrum or a high energy spectrum) includes an energy spectrum in an excess region exceeding the energy region of irradiated X-rays, the signal processing unit 133 can perform correction (pile-up correction) for excluding the X-ray spectrum in the excess region. For example, if the information of a spectrum counted in an excess region exceeding the upper limit or lower limit of the energy region of irradiated X-rays is included, the signal processing unit 133 performs pile-up correction of excluding the energy spectrum in the excess region, which is the information of the spectrum counted in the excess region, from the X-ray spectrum as an error generated by pile-up of the X-ray photon detector. Note that the above-described smoothing correction may be performed after pile-up correction.

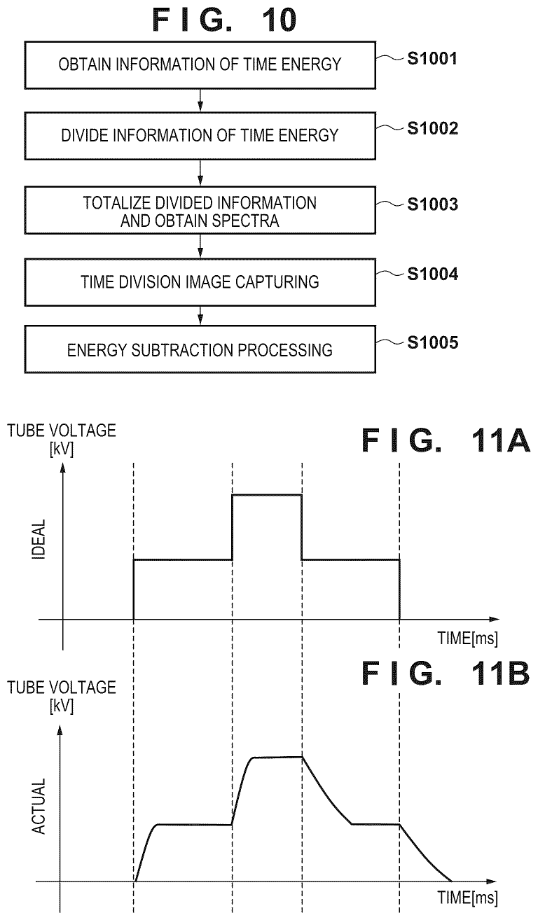

FIG. 10 is a flowchart showing the procedure of processing of the X-ray imaging system according to the first embodiment. The flowchart of FIG. 10 shows an example in which X-ray spectrum obtaining described with reference to FIGS. 8A to 8C and 9 is performed before image capturing. Note that the timing of X-ray spectrum obtaining is not limited to that in the procedure of processing shown in FIG. 10.

(S1001: Obtaining of Information of Time X-Ray Photon Energy)

In step S1001, the X-ray photon detector obtains the information of time X-ray photon energy (time series X-ray photon energy).

(S1002: Division of Information of Time X-Ray Photon Energy)

Next, in step S1002, the signal processing unit 133 divides the information of the time X-ray photon energy obtained by the X-ray photon detector. The signal processing unit 133 divides the information of the time X-ray photon energy into the X-ray photon groups 804, 805, and 806 in synchronism with, for example, the timings of sample and hold operations (SH_N and SH_S), as shown in FIG. 8B. The X-ray photon groups 804 and 806 correspond to the low energy sections 811 and 813 of convex X-ray pulses, and the X-ray photon group 805 corresponds to the high energy section 812 of convex X-ray pulses.

(S1003: Totalization and Obtaining of Plurality of Spectra)

In step S1003, the signal processing unit 133 totalizes the divided information of X-ray photon energy, thereby obtaining a plurality of spectra of different X-ray energies. Using the information of X-ray photon energy of the X-ray photon groups 804 and 806 corresponding to the low energy sections 811 and 813 and the information of X-ray photon energy of the X-ray photon group 805 corresponding to the high energy section 812, the signal processing unit 133 obtains, for example, the X-ray spectrum 807 of low energy and the X-ray spectrum 808 of high energy as shown in FIG. 8C or 9. The signal processing unit 133 then stores the obtained data of the plurality of spectra of different X-ray energies in a memory provided in the imaging control apparatus 103.

Here, of the obtained data of the plurality of spectra, for example, the X-ray spectrum 807 of low energy as shown in FIG. 8C or 9 corresponds to the spectrum N_{L}(E) of low energy X-rays in equations (4), and the X-ray spectrum 808 of high energy corresponds to the spectrum N_{H}(E) of high energy X-rays in equations (4).

(S1004: Time Division Imaging)

In step S1004, image obtaining is performed by time division imaging using the X-ray imaging system. The signal processing unit 133 of the X-ray imaging system performs radiation irradiation and imaging by, for example, time division imaging based on the timing chart shown in FIG. 4 or 9 and obtains a plurality of images (sub-images: H and L) corresponding to a plurality of different radiation energies.

(S1005: Energy Subtraction Processing)

In step S1005, the signal processing unit 133 performs energy subtraction processing using the plurality of images (sub-images: H and L) corresponding to the plurality of radiation energies obtained in step S1004 and the data of the plurality of spectra of different X-ray energies stored in the memory in step S1003.

When simultaneous equations (4) are solved using the sub-image 405 and the X-ray spectrum 807, and the sub-image 406 and the X-ray spectrum 808, an accurate energy subtraction image can be obtained. For example, the bone thickness image B and the soft tissue thickness image S which are accurate can be obtained as material decomposition images.

Note that the procedure up to the obtaining of the X-ray spectrum may be performed at a stage before providing to a customer. For example, the service department may obtain the data and store it in the memory in advance before shipment. X-ray spectrum data of a plurality of patterns may be prepared by changing the imaging conditions and stored in the memory, and the X-ray spectrum data may be changed in accordance with the imaging conditions. As the imaging conditions, for example, X-ray irradiation conditions (for example, a tube voltage, a tube current, and an accumulation time) assumed to be used by the customer or sample and hold conditions are preferably covered. As a change example of imaging conditions, not only the X-ray irradiation conditions and the sample and hold conditions but also various conditions may be set.

According to this embodiment, the energy spectrum of irradiated radiation can be obtained. When the obtained energy spectrum is used for energy subtraction processing, the accuracy of processing can be improved.

### SECOND EMBODIMENT

In the second embodiment, a configuration for optimizing imaging conditions will be described in addition to a configuration for time-dividing an X-ray spectrum. The configuration and driving of an X-ray imaging system according to the second embodiment are the same as in the first embodiment.

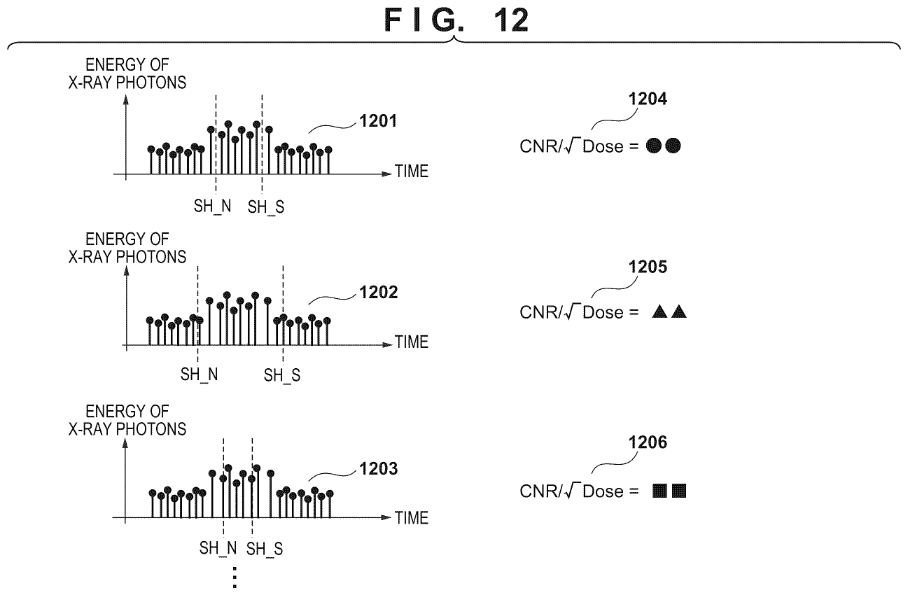

FIGS. 11A and 11B show an example of the waveform of X-rays to be explained in this embodiment. As the waveform of X-rays, processing according to this embodiment can be applied to various waveforms. Here, the waveform of convex X-rays will be described as an example. In convex X-rays, ideally, the voltage changes at a right angle at the timing of tube voltage switching, and otherwise, always takes a predetermined value, like a waveform indicated in FIG. 11A. The quality of an energy subtraction image in the X-ray imaging system depends on an X-ray energy difference ΔE and the dose ratio between sub-images. If a sample and hold operation is performed at the timing of voltage switching, the X-ray energy difference ΔE and the dose ratio can easily be obtained, and an energy subtraction image with assumed image quality can be obtained.

However, actually, the voltage never changes at a right angle at the timing of tube voltage switching, and the rising and falling of the voltage are blunt, like a waveform in FIG. 11B. In such a blunt waveform, it may be difficult to grasp the X-ray energy difference ΔE and the dose ratio between sub-images. In addition, since the X-ray energy difference ΔE and the dose ratio are largely changed only by changing the sample and hold timing by several ms, setting of the sample and hold timing may be difficult.

In this embodiment, the X-ray spectrum is time-divided, and the quality of an image obtained by energy subtraction processing at that time is simulated, thereby optimizing, for example, a sample and hold timing as an imaging condition. The simulation will be referred to as an image quality simulation hereinafter.

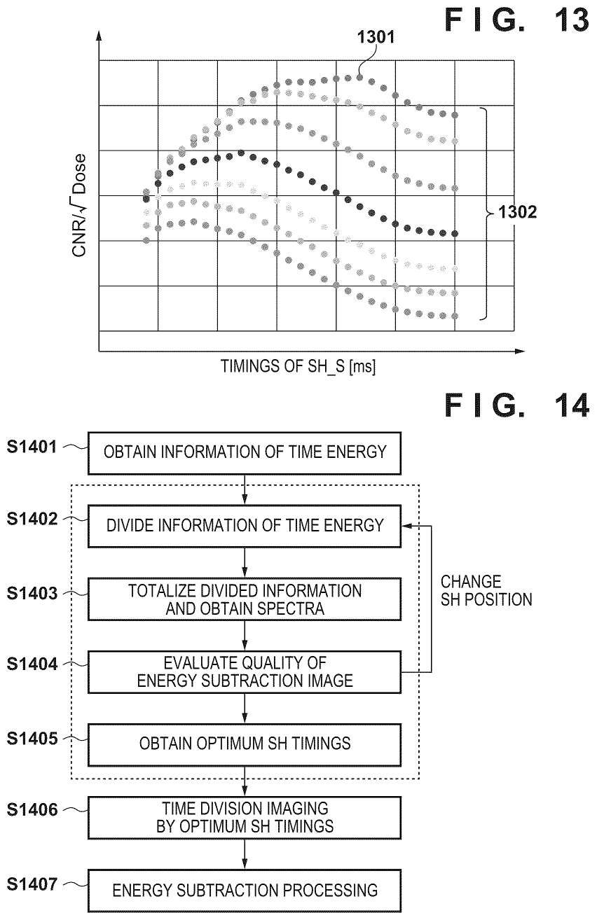

FIG. 12 shows the principle of the image quality simulation. In FIGS. 12, 1201 to 1203 indicate views showing the energy information of X-ray photons and sample and hold timings for which time is plotted along the abscissa, and the X-ray photon energy is plotted along the ordinate. The only differences between 1201 to 1203 are the sample and hold timings SH_N and SH_S indicated by dotted lines. In this embodiment, CNR/√Dose is used as evaluation information (index) representing the quality of an image obtained by the image quality simulation. CNR/√Dose is a value obtained by dividing the ratio of the contrast between a region where a target material exists and a region where no target material exists in an image obtained by energy subtraction processing to noise (contrast to noise ratio: CNR) by the square root of the dose. CNR/√Dose can show the visibility of the target material and can suitably be used as the index of an energy subtraction image. Here, the target material is a material contained in an object and indicates a material (the material can include, for example, a bone and a soft tissue) to be decomposed by the energy subtraction processing.

Referring to FIG. 12, pieces of evaluation information 1204 to 1206 represent the qualities of images obtained by energy subtraction processing using a plurality of sub-images (two images, that is, a high energy image H and a low energy image L) obtained under the imaging conditions of 1201 to 1203.

If the energy information of X-ray photons exists, not only the X-ray spectrum of a sub-image upon changing the sample and hold timing but also information such as the number of photons of X-rays and the X-ray energy can be obtained. When obtaining the evaluation information (CNR/√Dose), noise (N) and the dose can be obtained from the number of photons and energy of X-rays. As the contrast, the contrast between a region where a target material exists and a region where no target material exists in an image obtained by substituting the information of the X-ray spectrum and the X-ray energy to equations (4) and thus solving the simultaneous equations can be obtained. A linear attenuation coefficient μ B (E), a linear attenuation coefficient μ_{S}(E) of the soft tissue, and the types of thicknesses of materials such as a bone thickness B and a soft tissue thickness S in equations (4) can arbitrarily be changed in accordance with the object as the target of imaging.

Thus, if the energy information of X-ray photons exists, the quality of an image obtained by energy subtraction processing can be simulated. As the imaging condition, the sample and hold timing is changed like, for example, 1201 to 1203 in FIG. 12, and the evaluation information CNR/√Dose is calculated (for example, 1204 to 1206). If the imaging condition that maximizes the evaluation information CNR/√Dose is used, the sample and hold timing can be optimized, and an energy subtraction image with excellent image quality can thus be obtained.

FIG. 13 shows the correlation between the sample and hold timing obtained by the simulation and the quality of an image obtained by energy subtraction processing. The abscissa represents the timing of SH_S, and the ordinate represents the evaluation information CNR/√Dose. Sequences 1302 of the graph are different timings of SH_N. The condition of a maximum value 1301 of evaluation information in the ordinate direction is the optimum condition of sample and hold (SH_N and SH_S). When image obtaining is performed at the sample and hold timing corresponding to the maximum value 1301 of the evaluation information and a time-divided X-ray spectrum obtained by the method described in the first embodiment is used, an image with excellent image quality can be obtained by energy subtraction processing.

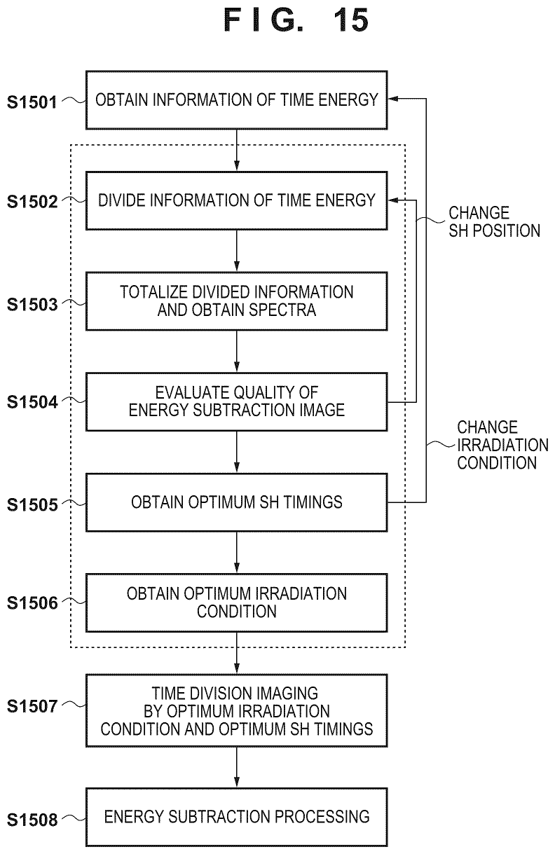

FIG. 14 is a flowchart of optimization processing of a sample and hold timing in the X-ray imaging system according to the second embodiment. In FIG. 14, processes of steps S1402 to S1405 surrounded by a broken line are processes by a simulation.

(S1401: Obtaining of Information of Time X-Ray Photon Energy)

In step S1401, an X-ray photon detector obtains the information of time X-ray photon energy (time series X-ray photon energy).

(S1402: Division of Information of Time X-Ray Photon Energy and Change of Division Position)

Next, in step S1402, a signal processing unit 133 divides the information of the time X-ray photon energy obtained by the X-ray photon detector at the set timings of sample and hold timing operations (SH_Nᵢ and SH_Sⱼ). Here, the subscripts i and j are parameters for changing the setting of sample and hold in iterative processing. If the parameters are changed like i=i+1 . . . , j=j+1 . . . , the timings of the sample and hold operations (SH_Nᵢ and SH_Sⱼ) can be changed. For example, if the parameter i is changed in the iterative processing, the setting of SH_N is changed for 1302 in FIG. 13. If the parameter j is changed, the setting of SH_S is changed for the abscissa direction in FIG. 13.

The signal processing unit 133 divides the information of the time X-ray photon energy into X-ray photon groups (for example, 804, 805, and 806) based on the set timings of sample and hold operations (SH_Nᵢ and SHⱼ).

(S1403: Totalization and Obtaining of Plurality of Spectra)

In step S1403, the signal processing unit 133 totalizes the divided information of X-ray photon energy, thereby obtaining a plurality of spectra of different X-ray energies. The signal processing unit 133 then stores the obtained data of the plurality of spectra of different X-ray energies in a memory provided in an imaging control apparatus 103.

(S1404: Image Quality Evaluation)

In step S1404, imaging is performed by radiation irradiation by time division imaging using the X-ray imaging system, thereby obtaining a plurality of images (sub-images) corresponding to a plurality of different radiation energies. The signal processing unit 133 then performs energy subtraction processing using the plurality of obtained images (sub-images: H and L) and the data of the plurality of spectra of different X-ray energies, calculates the evaluation information of the obtained image (energy subtraction image), and stores it in the memory.

The signal processing unit 133 repetitively executes the processes of steps S1402 to S1404 while changing the setting of the sample and hold operations (SH_N, and SH_Sⱼ). Based on the sample and hold operations (SH_N, and SH_Sⱼ) of the different setting, the signal processing unit 133 divides the information of the time X-ray photon energy, totalizes the divided information of the X-ray photon energy, and obtains a plurality of spectra of different X-ray energies. The signal processing unit 133 then performs energy subtraction processing, calculates the evaluation information of the obtained image (energy subtraction image), and stores it in the memory.

(S1405: Obtaining of Optimum Sample and Hold Timing)

In step S1405, the signal processing unit 133 sets, as the imaging condition, a timing at which the evaluation information takes the maximum value by changing the timing of sampling and holding the signal of X-ray energy. The signal processing unit 133 compares the plurality of pieces of evaluation information obtained by repetitively executing the processes of steps S1402 to S1404 and obtains a sample and hold timing at which the evaluation information CNR/√Dose is maximum as the optimum sample and hold timing. Time division imaging (step S1406) to be described below is performed using the optimum sample and hold timing obtained by the simulation.

(S1406: Time Division Imaging)

In step S1406, image obtaining is performed by time division imaging using the X-ray imaging system based on the optimum sample and hold timing. The X-ray imaging system performs imaging by time division imaging by performing radiation irradiation, and the signal processing unit 133 obtains a plurality of images (sub-images) corresponding to a plurality of different radiation energies.

(S1407: Energy Subtraction Processing)

In step S1407, the signal processing unit 133 performs energy subtraction processing using the plurality of images (sub-images) corresponding to the plurality of radiation energies and the data of the plurality of spectra of different X-ray energies obtained at the optimum sample and hold timing. When simultaneous equations (4) are solved using an attenuation rate image L (sub-image) at low energy and the X-ray spectrum of low energy, and an attenuation rate image H (sub-image) at high energy and the X-ray spectrum of high energy, an accurate energy subtraction image can be obtained. For example, the bone thickness image B and the soft tissue thickness image S which are accurate can be obtained as material decomposition images.

According to this embodiment, the energy spectrum of irradiated radiation can be obtained. When the obtained energy spectrum is used for energy subtraction processing, the accuracy of processing can be improved.

### THIRD EMBODIMENT

In the third embodiment, a configuration will be described, which time-divides an X-ray spectrum and simulates the quality of an image obtained by energy subtraction processing at that time, thereby optimizing, as imaging conditions, a sample and hold timing and an X-ray irradiation condition. The configuration and driving of an X-ray imaging system according to the third embodiment are the same as in the first embodiment, and image quality evaluation of an image obtained by energy subtraction processing and the configuration of simulation are the same as in the second embodiment.

FIG. 15 is a flowchart of optimization processing of a sample and hold timing and an X-ray irradiation condition in the X-ray imaging system according to the third embodiment. The difference from the flowchart of FIG. 14 is that a loop for changing the X-ray irradiation condition is added. In the processing procedure shown in FIG. 15, the X-ray irradiation condition is optimized in addition to the sample and hold timing. In FIG. 15, processes of steps S1502 to S1506 surrounded by a broken line are processes by a simulation.

(S1501: Obtaining of Information of Time X-Ray Photon Energy)

In step S1501, an X-ray photon detector obtains the information of time X-ray photon energy (time series X-ray photon energy). Here, an X-ray irradiation condition (k) in an X-ray generation apparatus 101 is set. Here, k is a parameter for changing the setting of the X-ray irradiation condition in iterative processing. If the parameter is changed like k=k+1 . . . , the X-ray irradiation condition can be changed.

(S1502: Division of Information of Time X-Ray Photon Energy and Change of Division Position)

Next, in step S1502, a signal processing unit 133 divides the information of the time X-ray photon energy obtained by the X-ray photon detector at the set timings of sample and hold timing operations (SH_Nᵢ and SH_Sⱼ).

(S1503: Totalization and Obtaining of Plurality of Spectra)

In step S1503, the signal processing unit 133 totalizes the divided information of X-ray photon energy, thereby obtaining a plurality of spectra of different X-ray energies. The signal processing unit 133 then stores the obtained data of the plurality of spectra of different X-ray energies in a memory provided in an imaging control apparatus 103.

(S1504: Image Quality Evaluation)

In step S1504, imaging is performed by radiation irradiation by time division imaging using the X-ray imaging system, thereby obtaining a plurality of images (sub-images) corresponding to a plurality of different radiation energies. The signal processing unit 133 then performs energy subtraction processing using the plurality of obtained images (sub-images: H and L) and the data of the plurality of spectra of different X-ray energies, calculates the evaluation information of the obtained image (energy subtraction image), and stores it in the memory.

The signal processing unit 133 repetitively executes the processes of steps S1502 to S1504 while changing the setting of the sample and hold operations (SH_N, and SH_Sⱼ).

(S1505: Obtaining of Optimum Sample and Hold Timing)

In step S1505, the signal processing unit compares the plurality of pieces of evaluation information obtained by repetitively executing the processes of steps S1502 to S1504 and obtains a sample and hold timing at which evaluation information CNR/√Dose is maximum as the optimum sample and hold timing.

The signal processing unit 133 returns the process to step S1501 and repeats the same processing as described above after changing the X-ray irradiation condition (k). The signal processing unit 133 sets a sample and hold timing at which the evaluation information CNR/√Dose is maximum, calculates the evaluation information CNR/√Dose under the condition of the changed X-ray irradiation condition (k), and stores it in the memory.

(S1506: Obtaining of Optimum X-Ray Irradiation Condition)

In step S1506, the signal processing unit 133 evaluates, based on the evaluation information, the quality of an image obtained by the simulation of energy subtraction processing under the changed imaging condition, and sets an imaging condition with which the evaluation information takes the maximum value. In this step, the signal processing unit 133 changes the X-ray irradiation condition as the imaging condition, and sets an irradiation condition with which the evaluation information takes the maximum value. The signal processing unit 133 repetitively executes the processing of calculating the evaluation information CNR/√Dose under the condition of the changed X-ray irradiation condition, compares the plurality of pieces of obtained evaluation information, and obtains an X-ray irradiation condition (k) at which the evaluation information CNR/√Dose is maximum as the optimum X-ray irradiation condition. Time division imaging (step S1507) to be described below is performed using the optimum sample and hold timing obtained by the simulation (step S1505) and the optimum X-ray irradiation condition (step S1506).

(S1507: Time Division Imaging)

In step S1507, time division imaging is performed under the optimum imaging conditions (the X-ray irradiation condition and the sample and hold timing). That is, based on the optimum X-ray irradiation condition and the optimum sample and hold timing, image obtaining is performed by time division imaging using the X-ray imaging system. The X-ray imaging system performs imaging by time division imaging by performing radiation irradiation, and the signal processing unit 133 obtains a plurality of images (sub-images) corresponding to a plurality of different radiation energies.

(S1508: Energy Subtraction Processing)

In step S1508, the signal processing unit 133 performs energy subtraction processing using the plurality of images (sub-images) corresponding to the plurality of radiation energies and the data of the plurality of spectra of different X-ray energies obtained under the optimum imaging conditions (the X-ray irradiation condition and the sample and hold timing).

When simultaneous equations (4) are solved using an attenuation rate image L (sub-image) at low energy and the X-ray spectrum of low energy, and an attenuation rate image H (sub-image) at high energy and the X-ray spectrum of high energy, an accurate energy subtraction image can be obtained. For example, a bone thickness image B and a soft tissue thickness image S which are accurate can be obtained as material decomposition images.

According to this embodiment, the energy spectrum of irradiated radiation can be obtained. When the obtained energy spectrum is used for energy subtraction processing, the accuracy of processing can be improved, and an energy subtraction image with excellent image quality can be obtained.

(Modifications)

In the first to third embodiments, the X-ray imaging apparatus 104 is an indirect type X-ray sensor using a phosphor. However, the disclosed technique is not limited to this form. For example, a direct type X-ray sensor using a direct conversion material such as CdTe may be used.

In the first to third embodiments, a case where as the X-ray energies, two high- and low-level X-ray energies, including a first energy level and a second energy level higher than the first energy level are used has been described. However, the disclosed technique is not limited to these embodiments. For example, the technique can also be applied to a case where the number of levels of X-ray energies are three or more.

In the first to third embodiments, the X-ray energy is changed, thereby obtaining an image of a different energy. However, the disclosed technique is not limited to this form. A plurality of phosphors 105 and a plurality of two-dimensional detectors 106 may be overlaid, and images of different energies may be obtained from the two-dimensional detector on the front side and the two-dimensional detector on the rear side with respect to the direction of incidence of X-rays. The two-dimensional detector 106 is not limited to a medical device, and an industrial two-dimensional detector may be used.

In the first to third embodiments, energy subtraction processing is performed using the imaging control apparatus 103 of the radiation imaging system. However, the disclosed technique is not limited to this form. An image obtained by the imaging control apparatus 103 may be transferred to another computer, and energy subtraction processing may be performed. For example, an obtained image may be transferred to another personal computer (image viewer) via a medical PACS, and the processing result may be displayed on the radiation imaging system after energy subtraction processing is performed.

In the first to third embodiments, an example of convex X-rays in a case where the tube voltage of X-rays is actively switched has been described. However, the embodiment is not limited to this example, and the tube voltage may be switched, for example, stepwise. The number of steps may be two, or three or more. Also, the tube voltage may be switched to increase like an ascending staircase, or the tube voltage may be switched to decrease like a descending staircase. For example, if the tube voltage increases like an ascending staircase, the X-ray generation apparatus 101 generates X-rays by switching at least a first tube voltage and a second tube voltage higher than the first tube voltage. If the tube voltage decreases like a descending staircase, the X-ray generation apparatus 101 generates X-rays by switching at least the second tube voltage and the first tube voltage.

Sample and hold operations or time division of X-ray photon energy may be increased/decreased in accordance with the number of steps of the staircase. For example, as described above with reference to FIG. 4, the highest tube voltage may be set to the median, the tube voltage on the left side of the median may be set to increase stepwise, and the tube voltage on the right side of the median may be set to decrease stepwise.

Also, in the first to third embodiments, the tube voltage of the X-ray generation apparatus 101 (radiation generation apparatus) is changed. However, the disclosed technique is not limited to this form. The energy of X-rays with which the X-ray imaging apparatus 104 is irradiated may be changed by, for example, temporally switching the filter of the X-ray generation apparatus 101.

In the first to third embodiments, a medical use such as decomposition of a bone and a soft material has been described as an example. However, the disclosed technique is not limited to this form. For example, application to an industrial use such as defect inspection for circuit boards is also possible.

In the second and third embodiments, CNR/√Dose is used as an index for evaluating the quality of an energy subtraction image. However, the disclosed technique is not limited to the embodiments using this index. For example, CNR may be used as the index without taking the dose into consideration. Alternatively, a parameter other than an image quality such as the X-ray energy difference ΔE or dose ratio between the sub-images (the image H and the image L) may be used as the index. For example, the signal processing unit 133 may set an imaging condition with which evaluation information using the energy difference of X-ray energies irradiated when obtaining the plurality of images (the image H and the image L) or evaluation information using the dose ratio of X-rays irradiated when obtaining the plurality of images takes the maximum value.

According to the disclosed technique, the energy spectrum of irradiated radiation can be obtained. When the obtained energy spectrum is used for energy subtraction processing, the accuracy of processing can be improved.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-095224, filed Jun. 13, 2022 which is hereby incorporated by reference herein in its entirety.

What is claimed is:

1. A radiation imaging apparatus comprising:
a processing unit configured to obtain a plurality of images corresponding to a plurality of different radiation energies by irradiating an object with radiation and performing imaging using an energy spectrum obtained by totaling energy information obtained by dividing a time-serially obtained radiation photon energy in a time direction, and perform energy subtraction processing using the plurality of images.

2. The apparatus according to claim 1, wherein the processing unit obtains the energy spectrum by totalizing the energy information obtained by dividing the radiation photon energy at a timing of sampling and holding a signal of the radiation energy.

3. The apparatus according to claim 1, wherein the processing unit obtains the energy spectrum by counting the number of photons in time series in the energy information.

4. The apparatus according to claim 1, wherein the processing unit divides the radiation photon energy at a timing of sampling and holding a signal corresponding to a first radiation energy and at a timing of sampling and holding a signal corresponding to a second radiation energy higher than the first radiation energy.

5. The apparatus according to claim 1, further comprising an obtaining unit configured to time-serially obtain the radiation photon energy when radiation irradiation is performed at least once or a plurality of times,
wherein if the radiation irradiation is performed a plurality of times, the processing unit totalizes, for each energy of the radiation, the radiation photon energy divided at a timing of sampling and holding a signal corresponding to the radiation energy, thereby obtaining the energy information.

6. The apparatus according to claim 5, wherein if the radiation irradiation is performed a plurality of times, the processing unit divides the radiation photon energy at a timing matching each irradiation.

7. The apparatus according to claim 1, further comprising an obtaining unit configured to obtain the radiation photon energy in synchronism with radiation irradiation based on a temporally changing tube voltage.

8. The apparatus according to claim 1, wherein if the energy spectrum includes an energy spectrum in an excess region exceeding an energy region of radiation, the processing unit performs pile-up correction for excluding the energy spectrum in the excess region.

9. The apparatus according to claim 1, wherein the processing unit performs smoothing correction for correcting a waveform of the energy spectrum to a smooth shape.

10. The apparatus according to claim 1, further comprising a radiation generation unit configured to generate radiation by switching a tube voltage,
wherein the radiation generation unit
generates the radiation by switching a first tube voltage, a second tube voltage higher than the first tube voltage, and the first tube voltage, or
generates the radiation by switching at least the first tube voltage and the second tube voltage, or
generates the radiation by switching at least the second tube voltage and the first tube voltage.

11. The apparatus according to claim 2, wherein the processing unit evaluates, based on evaluation information, quality of an image obtained by a simulation of the energy subtraction processing under a changed imaging condition, and sets an imaging condition with which the evaluation information takes a maximum value.

12. The apparatus according to claim 11, wherein the processing unit changes, as the imaging condition, the timing of sampling and holding the signal of the radiation energy and sets the timing at which the evaluation information takes the maximum value.

13. The apparatus according to claim 11, wherein the processing unit changes, as the imaging condition, an irradiation condition of the radiation and sets the irradiation condition under which the evaluation information takes the maximum value.

14. The apparatus according to claim 11, wherein the processing unit sets the imaging condition under which
evaluation information using a ratio of a contrast to noise of the image or
evaluation information using a value obtained by dividing the ratio of the contrast and the noise by a square root of a dose of the radiation takes the maximum value.

15. The apparatus according to claim 11, wherein the processing unit sets the imaging condition under which
evaluation information using an energy difference between the plurality of radiation energies irradiated when obtaining the plurality of images or
evaluation information using a dose ratio of the radiation irradiated when obtaining the plurality of images takes the maximum value.

16. An information processing apparatus comprising:
a processing unit configured to obtain a plurality of images corresponding to a plurality of different radiation energies by irradiating an object with radiation and performing imaging using an energy spectrum obtained by totaling energy information obtained by dividing a time-serially obtained radiation photon energy in a time direction, and perform energy subtraction processing using the plurality of images.

17. An information processing method comprising:
obtaining a plurality of images corresponding to a plurality of different radiation energies by irradiating an object with radiation and performing imaging using an energy spectrum obtained by totaling energy information obtained by dividing a time-serially obtained radiation photon energy in a time direction, and performing energy subtraction processing using the plurality of images.

18. A non-transitory computer-readable storage medium storing a program for causing a computer to execute the method according to claim 17.
